(19) United States
(12) Patent Application Publication
(10) Pub. No.: US 2024/0000346 A1
(43) Pub. Date: Jan. 4, 2024

(54) FUNCTIONING OF INTERSTITIAL FLUID HARVESTING AND PROCESSING PATCH USING GEL OSMOSIS AND PAPER MICROFLUIDICS

(71) Applicant: North Carolina State University, Raleigh, NC (US)

(72) Inventors: Tamoghna Saha, Raleigh, NC (US); Orlin D. Velev, Raleigh, NC (US); Michael D. Dickey, Raleigh, NC (US); Michael Daniele, Raleigh, NC (US)

(21) Appl. No.: 18/217,392

(22) Filed: Jun. 30, 2023

Related U.S. Application Data
(60) Provisional application No. 63/357,569, filed on Jun. 30, 2022.

(51) Int. Cl. A61B 5/145; A61B 5/1473

(52) U.S. Cl. CPC A61B 5/14514; A61B 5/14532; A61B 5/14539; A61B 5/14546; A61B 5/1473; A61B 2505/07; A61B 2560/063

(57) ABSTRACT

Various examples are provided related to interstitial fluid (ISF) or extracellular fluid (ECF) harvesting and processing. In one example, a microfluidic monitoring platform includes a microneedle patch including microneedles on a first side; an osmotic patch on a second side of the microneedle patch that includes glycerogel or hydrogel equilibrated with glycerin or glucose; and a microfluidic or fluid transport film or material channel between the osmotic patch and the microneedle patch. The channel can extract fluid from the osmotic-microneedle patch complex. In another example, a wearable electrochemical sensing system includes a monitoring platform including a microneedle-osmotic patch, a microfluidic or fluid transport film or material channel, and at least one sensor between the channel; and processing circuitry coupled to the at least one sensor. The processing circuitry can monitor presence of a chemical or biomarker in the fluid based upon signals obtained from the sensor.

### CROSS REFERENCE TO RELATED APPLICATIONS

This application claims priority to, and the benefit of, co-pending U.S. provisional application entitled “Functioning of Interstitial Fluid Harvesting and Processing Patch Using Gel Osmosis and Paper Microfluidics” having Ser. No. 63/357,569, filed Jun. 30, 2022, which is hereby incorporated by reference in its entirety.

### STATEMENT REGARDING FEDERALLY SPONSORED RESEARCH OR DEVELOPMENT

This invention was made with government support under grant number EEC1160483 awarded by the National Science Foundation. The government has certain rights in the invention.

### BACKGROUND

Interstitial fluid (ISF) contains diverse biomarkers that provide essential information regarding the health and physiological state of a human body. ISF is a thin layer of fluid which surrounds the body cells and contains dissolved substances that are exchanged with blood capillaries. It also helps in bringing oxygen and nutrients to cells and removing waste products from them.

### SUMMARY

Aspects of the present disclosure are related to interstitial fluid (ISF) or extracellular fluid (ECF) harvesting and processing. In one aspect, among others, a microfluidic monitoring platform, comprises a microneedle patch comprising a plurality of microneedles on a first side; an osmotic patch disposed on a second side of the microneedle patch opposite the plurality of microneedles, the osmotic patch comprising glycerogel or hydrogel or ionogel or other solid electrolyte equilibrated with glycerin, glucose, or other osmolyte; and a microfluidic or fluid transport film or material channel disposed between the osmotic patch and the microneedle patch, the paper or woven mat, non-woven membrane, or film-based microfluidic channel configured to extract fluid through the microneedle patch via capillary wicking. In one or more aspects, the glycerogel can comprise pure glycerin. The fluid can be interstitial fluid (ISF) or extracellular fluid (ECF).

The osmotic patch generates as chemical potential difference with the hydrate microneedle patch to transport the ISF out of the microneedle patch. And the interface of the osmotic patch is volume constrained.

In various aspects, the microfluidic or fluid transport film or material channel can comprise an extraction portion, a transport portion, and an evaporation portion. The extraction portion can comprise a circular paper pad. The circular paper pad can comprise a pattern of openings. The transport portion can comprise a rectangular section extending between the extraction portion and the evaporation portion. The rectangular section can comprise a linear path or a tortuous path. The evaporation portion can comprise an evaporation pad at an end of the rectangular section.

In another aspect, a wearable electrochemical, optical, electrical, colorimetric, and/or photonic sensing system comprises a microfluidic monitoring platform comprising: an osmotic patch comprising glycerogel or hydrogel; a paper or woven mat, non-woven membrane, or film-based microfluidic channel configured to extract fluid through the microneedle patch via capillary wicking; and at least one sensor disposed between the osmotic patch and the microfluidic or fluid transport film or material channel; and flexible wireless processing circuitry coupled to the at least one sensor, the processing circuitry configured to monitor presence of a chemical or biomarker in the fluid based upon signals obtained from the sensor. In one or more aspects, the at least one sensor can comprise functionalized sensing electrodes.

In various aspects, the fluid can be interstitial fluid (ISF) or extracellular fluid (ECF). The hydrogel can be equilibrated with glycerin or glucose. The processing circuitry can comprise potentiostat circuitry, analog front-end, and optoelectronic systems. The processing circuitry can comprise a communications interface configured for wirelessly communicate sensor information to a user device. The microfluidic or fluid transport film or material channel can comprise an extraction portion, a transport portion, and an evaporation portion. The extraction portion can comprise a circular paper pad including a pattern of openings. Electrodes of the at least one sensor can be located between the openings of the circular paper pad. The transport portion can comprise a rectangular section having a tortuous path.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a microfluidic monitoring platform comprising a microneedle patch, osmotic patch, and microfluidic channel, in accordance with various embodiments of the present disclosure.

FIGS. 2A and 2B illustrate qualitative analysis of functioning of the integrated platform using different osmolytes, in accordance with various embodiments of the present disclosure.

FIGS. 3A-3C illustrate examples of normalized cumulative dye intensity and quantity of dye sampled on evaporation pad and paper channel with different gel systems, in accordance with various embodiments of the present disclosure.

FIGS. 4A-4F illustrate an example of an osmotic wearable system for continuous biochemical monitoring, in accordance with various embodiments of the present disclosure.

FIGS. 5A-5D illustrate an example of a flexible wearable potentiostat (FWP), in accordance with various embodiments of the present disclosure.

FIG. 6 illustrates an example of a wearable electrochemical sensing system including the patch with a paper microfluidic channel with a smaller evaporation pad, in accordance with various embodiments of the present disclosure.

### DETAILED DESCRIPTION

Disclosed herein are various examples related to interstitial fluid (ISF) or extracellular fluid (ECF) harvesting and processing. Utilizing gel osmosis and paper microfluidics offers significant benefits for ISF extraction. A microfluidic monitoring platform comprising a microneedle patch, an osmotic patch and a paper microfluidic channel or conduit is presented. The osmotic patch can include glyerogel or hydrogel equilibrated with glycerin or glucose to increase the osmotic strength. Wearable and wireless monitoring of biomarkers can be integrated with the monitoring platform. Reference will now be made in detail to the description of the embodiments as illustrated in the drawings, wherein like reference numbers indicate like parts throughout the several views.

Microneedle patches have gained an increasing attention and represent a growing field of research since they hold the potential to sample ISF in a minimally invasive and less painful manner. Glucose, lactate, alcohol, and drugs are a few biomarkers that have been analyzed from the ISF using microneedles. Out of all biomarkers, it is seen that the glucose levels in ISF correlate well with that of blood. Microneedles have also been used to perform transdermal drug delivery.

Sampling of biomarkers from ISF using microneedle patches have been conducted only by directly interfacing them onto the skin surface. As a result, these patches withdraw fluid for a short time (about a few minutes) and sample extremely low fluid volume (about a μL). These patches lack a component for supporting long-term ISF collection and management, and thus fail to preserve a long-term record of the biomarker. Additionally, quantifying biomarkers from such low fluid volumes can raise questions concerning its clean capture, external contamination, and losses due to evaporation. Thus, there exists a need for a technique that would facilitate long-term ISF sampling and deliver a cumulative record of the biomarkers.

Human ISF has an ionic strength of about 0.15M and hence, in principle, the difference in the chemical potential due to solute concentration may be utilized as a driving force to withdraw it non-invasively. Osmotic pressure is a natural phenomenon that results from solute-solvent chemical potential imbalance between two solutions, separated by a semi-permeable membrane, which persists until the difference is annulled. Osmotic driven fluid transport has applications such as in-vitro extraction of several compounds, drug delivery, and in-vivo biomarker sampling.

The use of paper microfluidic channels can be an extremely simple and efficient approach for long-term ISF harvesting and management. Paper is not only a readily available and inexpensive material, but its porous nature and the ability to control flow rates via capillary wicking and evaporation, serves as some of its distinctive features as a medium to transport and manage ISF. Additionally, evaporation-assisted passive pumping has been used effectively by researchers to develop several microfluidic devices. Yet, the combined actions of passive osmotic microfluidic pumping and evaporation-assisted fluid management via paper have not been deployed in an operational wearable ISF monitoring platform for biomarker analysis.

In this disclosure, the in-vitro operation of an integrated prototype that uses hydrogel to interface with a microneedle patch for pumping ISF from model skin settings is presented. It includes the action of three basic effects: osmosis, capillary wicking through paper and evaporation for sampling ISF. The patches can sustain continuous, long-term (about hours to days) model ISF flow and biomarker sampling.

The operation of a long-term, continuous, and non-invasive platform for sampling of interstitial fluid (ISF) will be discussed. The platform can be composed of silicone, hydrogel patch, microneedle patch, and paper microfluidic conduit or channel with a site of the evaporation at the end (e.g., an evaporation pad). The hydrogel is equilibrated with either glycerin, or glucose to increase its osmotic strength to a high enough level relative to microneedles and ISF. This hydrogel can withdraw ISF and its associated biomarkers through the microneedle patch interfacing the skin. Benchtop experiments (with model biomarker and model skin) were performed to characterize the functioning of this platform. It was found that hydrogels treated with pure glycerin (“glycerogels”) worked best in sampling the maximum amount of model biomarker on the paper channel over a 5-hour period. Control experiments have shown that microneedles aid with fluid transfer across the hydrogel-skin interface. Such ISF sampling patches can provide useful information about ISF biomarker levels, especially glucose (since glucose in ISF correlates well with blood glucose) and contribute towards the development of wearable devices.

Microfluidic Monitoring Platform

Utilizing glycerogels with microneedles offers significant benefits for ISF extraction. Glycerogels possess a very high osmotic pressure relative to ISF and microneedles. A high osmotic pressure indicates a higher fluid withdrawing power. Current ISF sampling techniques with microneedle patches function briefly after interfacing the patch onto the skin. As a result, these patches withdraw ISF only for a short period of time (about a few minutes). Hence, when the glycerogel interfaces the microneedle patch and contacts the skin, prolonged (about hours) sampling of greater ISF volume can be achieved. Utilizing osmotic pressure difference via glycerogels to sample ISF is unique and has not been reported. Use of evaporation for sustaining continuous ISF collection is also beneficial. The sampled ISF flows through the rectangular section of the paper channel towards the evaporation pad. The ISF evaporates over time which maintains the capillary pressure in the paper channel to sustain continuous ISF collection. Utilizing evaporation along with osmosis for continuous ISF sampling has not been reported.

Osmotic Patch Fabrication. Sylgard-184 silicone elastomer (Dow Corning) and its curing agent were mixed in 10:1 w/w ratio and cured for 12 hours at 70° C. to make the base for the PDMS sheet. The patch was prepared by attaching two PDMS sheets together −38 mm×15 mm×2 mm (bottom) and 30 mm×15 mm×1 mm (top). A single hole matching the hydrogel diameter was punched at 6 mm from one of the edges on the bottom sheet to encase the hydrogel. A section of Whatman 542 paper was cut out using a CO 2 laser cutter (Universal Laser Systems VLS 3.5) and was sandwiched between the top and bottom PDMS sheets. The dimension of the paper channel determined based upon work on capillary assisted evaporative pumping. The PDMS sheets were attached together using additional silicone (Sylgard-184), making sure that it did not contact the paper channel. The whole patch was treated in an oven at 40° C. overnight to achieve firm adhesion between the sheets.

Hydrogel Synthesis. Hydrogel for the osmotic interface was made using acrylamide monomer, N—N′ methylenebisacrylamide as crosslinker and 2-hydroxy-4′-(2-hydroxyethoxy)-2-methylpropiophenone as the photo-initiator. The monomer solution contained 22% (w/w) acrylamide, 0.48% (w/w) crosslinker and 0.15% (w/w) photo-initiator. The solution was cured inside a circular petri dish (47 mm diameter) under a 175 mW/cm2 UV lamp (Sunray 400-SM) for three minutes. Disks of 8 mm diameter were punched out and stored in either 4M glucose solution 12M glycerin, 8M glycerin or pure glycerin for 24 hours. These solutions served as the osmolytes. Infusing the hydrogels with pure glycerin instead of water leads to a new class of gels called “glycerogels”. These gels majorly contain glycerin. After 24 hours, the infused disks were transferred to a fresh solution and stored for further usage. A single disk was taken out, blotted with a paper napkin, and was fitted inside the circular space in the patch before usage.

Microneedle Fabrication. Microneedles were fabricated using methacrylated hyaluronic acid (meHA). A solution of 4 wt % was prepared and added to clean, dry mold. It was then centrifuged at 3800×g for 4 minutes. The molds were then filled once again to create a backing layer and allowed to dry at ambient temperature. After allowing to dry completely in the molds, the microneedle patches were removed and crosslinked by exposing to 30 mW/cm2 UV for 300 seconds.

Fabrication of Model Skin. PBS was initially heated to about 80° C. 13% (w/w) gelatin type-A solution was prepared in 6 mL PBS solution. A dye solution (“model biomarker”) of 2.45% (v/v) of a yellow fluorescent dye in PBS was prepared and added to the gelatin-PBS mixture and stirred well. This gelatin-dye-PBS mixture was then transferred to a mold and was refrigerated overnight to obtain a solid gel-like material. This gel served as the “model skin”. For some experiments, a thin dialysis membrane (MWCO −12 KDa −14 KDa) was also placed over the top surface of the gel to mimic the tough semipermeable epidermal layer of skin and was altogether used as the model skin. The patch with an embedded hydrogel/glycerogel interfaced the microneedle patch, which itself interfaced the model skin. This served as the in-vitro (benchtop) experimental setup.

Image Analysis of Model Biomarker on Evaporation Pad. A test method was developed for quantitative measurement of the dye accumulated on the evaporation pad. A PDMS block (25 mm×25 mm) was placed separately on top of the hydrogel patch after interfacing with model skin to establish firm contact between the skin and hydrogel. The whole setup was covered with a rectangular petri dish with four square shaped side cutouts for continual fluid evaporation. The setup was then placed over a black cardboard and covered with a cardboard box to eliminate the illumination effects from the surrounding light. Six UV LED lights, from an ACLOROL 5050 LED strip, were placed approximately 2 inches in front of the paper strips to illuminate the fluorescent dye. A Canon EOS Mark5 DSLR camera was placed and focused on the patch from 15-30 cm away. Time lapse images of the dye flow were taken every 15 minutes until 6 hours. The test was performed under ambient lab conditions (about 22° C. and 40% RH) and was repeated three times.

MATLAB (R2018b) was used to analyze the accumulated dye profile on the evaporation pad. A small rectangular section on the pad (8 mm×2 mm) was selected each time for intensity measurements. Green channel intensity was calculated and averaged for pixels along a horizontal line that ran through the center of the selected section. Net intensity of the accumulated dye at a certain time (t) was calculated by subtracting its area under the curve from area under the curve at t=0. The difference was normalized on a 0-1 scale with 0 being the minimum and 1 being the maximum intensity.

Quantification of Model Biomarker on the Paper Channel, Glycerogel, and Microneedle Patch. After experiment, the whole paper channel was divided into the rectangular strip and evaporation pad through cutting. The strip, evaporation pad, hydrogel/glycerogel and the microneedle patch were individually stored in 5 mL of PBS solution overnight. This allowed the sampled dye to completely dissolve into the solution. The concentration of dye was quantified using a UV-vis spectrophotometer (Jasco UV/Vis V-550).

Testing and Evaluation

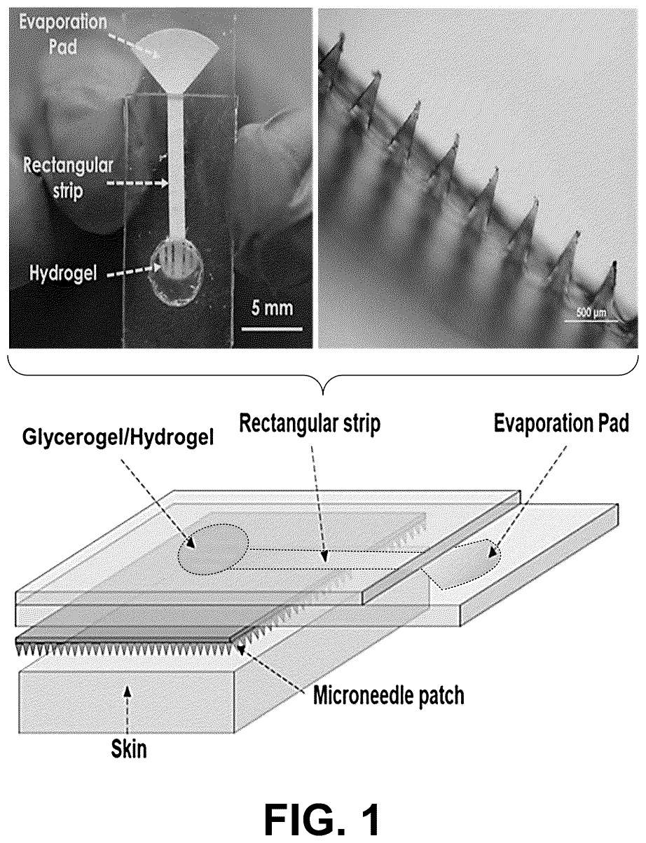

Referring to FIG. 1, shown is an example of an osmotic patch, a microneedle patch and the integrated benchtop experimental setup including a paper microfluidic channel between the microneedle patch and osmotic patch comprising hydrogel/glycerogel. The osmotic patch hosts hydrogel or glycerogel and interfaces the microneedle patch, which itself interfaces with the model skin. This experimental setup mimics the operation of this “integrated patch” (osmotic patch and microneedle patch) on real skin.

The osmotic pressure difference between the skin, microneedles, and either hydrogel or glycerogel, can be utilized to drive the flow of ISF from skin to microneedles and then eventually to the paper channel in the osmotic patch. Hydrogel disks were equilibrated with either glycerin solutions (which are benign to human skin) of varying concentration or pure glycerin (“glycerogel”), to lower their chemical potential (of the water in the gel) relative to ISF. Both the hydrogel and glycerogel serve as the main pumping source that facilitates long-term fluid withdrawal from the skin. The infused hydrogel/glycerogel is then loaded into the designated circular chamber of the patch. The hydrogel/glycerogel withdraws ISF and its associated biomarkers from the skin surface, through the microneedles due to a high osmotic pressure difference. The extracted ISF and biomarker (from skin), together with some of the osmolyte solution (from the hydrogel) get wicked through the rectangular paper strip towards the evaporation pad. The transported fluid mixture then reaches the evaporation pad, where it evaporates over time. Continuous evaporation of both the osmolyte and the ISF with biomarkers leads to their accumulation on the evaporation pad.

Due to the continuous fluid evaporation, the pad acts as a repository of any solute and biomarkers dissolved in the ISF. The evaporation of water from the pad maintains the capillary pressure in the paper strip of the microfluid channel to promote continuous long-term fluid flow towards the evaporation pad. The patch would cease to operate (stop withdrawing fluid) if either of the following conditions are reached over time: (1) chemical potential of the water in the ISF equals the chemical potential of water in the hydrogel or (2) saturation of the pad by deposited salt/osmolyte. Hence, the useful duration of patch operation depends on both the osmotic strength of the hydrogel/glycerogel and the dynamic free available surface area on the evaporation pad. This can be referred to as ‘dynamic’ due to the continuous deposition of biomarker and osmolyte over time.

Validation of Osmotic Pressure Difference. The role of hydrogel composition on dye and solute deposition levels on the pad was quantified. Initially, experiments were conducted similar to the ones in FIG. 1 for a 5-hour period. The hydrogel disks were first equilibrated with solutions of either, 12M glycerin, 8M glycerin, 4M glucose or pure glycerin. A control experiment was also conducted with glycerogel in the osmotic patch but without the microneedle patch.

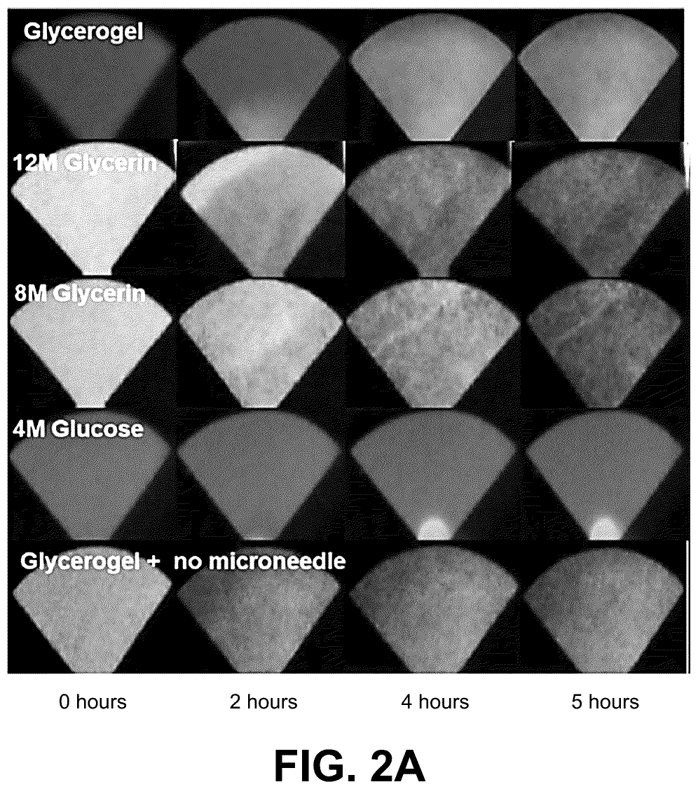

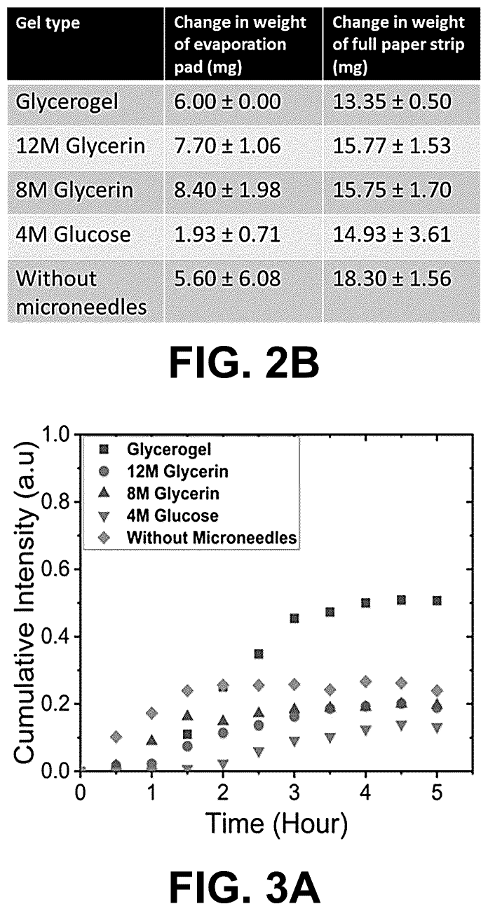

Upon interfacing the integrated patch to the model skin (containing yellow fluorescent dye), it was first observed that clear PBS fluid wicked through the paper channel and reached the end of the pad in 1-2 hours. The dye transport front lagged behind PBS and took about 3-4 hours to reach the end of the evaporation pad as shown in FIG. 2A, which illustrates examples of the qualitative analysis of the integrated patch (evaporation pad) functioning using different osmolytes. Chromatographic effects arising from the dye adsorption-desorption equilibria between the dye molecules in the PBS and paper substrate result in the lagging of dye. The total sampled fluid volume ranged from 5-10 μL on pad, while being from 15-20 μL on the paper strip. In FIG. 2A, the gels are ranked from best extraction (highest dye intensity) to worst: glycerogel, 12M glycerin, 8M glycerin and 4M glucose. Mass balance studies show that all type of gels sample similar quantity of fluid on the paper channel. However, 4M glucose hydrogel samples the least on the evaporation pad. The table of FIG. 2B summarizes the overall mass balance of the model skin with the integrated patch using different gel types after 5 hours of operation. The data represents the mean±the standard deviation (SD) from three trials.

The dye intensity levels on the evaporation pad depend strongly on the osmolyte being used to treat the hydrogel. Equilibration of the hydrogel with pure glycerin (glycerogel) causes the transfer of water from hydrogel to glycerin due to osmosis. As a result, the glycerogel becomes highly water depleted and possesses a very high osmotic strength. Once embedded in the integrated patch and made in contact with model skin, the glycerogel swells by the highest amount (change in weight of glycerogel about 130.85 mg) and leads to the largest amount of dye sampling on the paper channel until about 5 hours (see the first row of FIG. 2A). At 5 hours the evaporation pad was completely saturated with glycerin, which eventually terminates further fluid evaporation and dye collection.

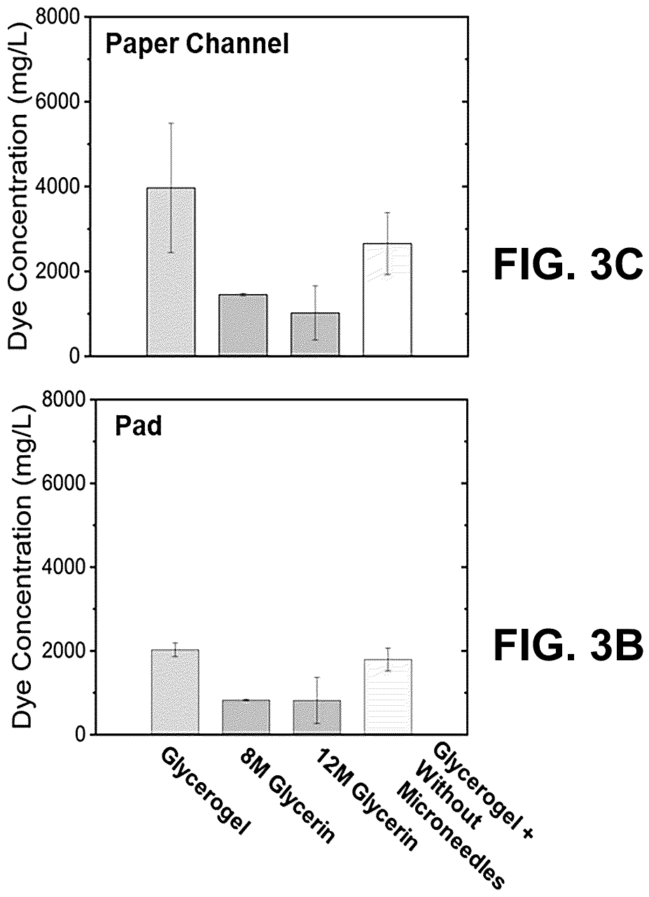

FIG. 3A illustrates examples of Normalized cumulative dye intensity measured on the evaporation pad over time for different gel systems. Note that the bottom row was without the use of microneedles and involved just the glycerogel. FIG. 3B illustrates the quantity of dye sampled on the evaporation pad by different gel systems and FIG. 3C illustrates the quantity of dye sampled on the full paper channel by different gel systems. The error bars denote the standard deviation (SD) from three trials. The intensity of the dye sampled by 12M glycerin and 8M glycerin hydrogel on the evaporation pad and paper channel are similar. The 12M glycerin hydrogel swells more than 8M glycerin hydrogel due to a higher osmotic strength. However, a greater inflow of glycerin towards the pad, stops fluid evaporation at a quicker rate in 12M glycerin hydrogels. This eventually reduces the inflow of dye towards the evaporation pad and makes the paper channel sample less dye. As a result, the difference between the amount of dye sampled by 12M glycerin and 8M glycerin hydrogel stays negligible. The 4M glucose hydrogel samples the least amount of dye and fluid on the evaporation pad. The inflowing glucose solution evaporates over time and deposits glucose at the junction between the rectangular strip and evaporation pad as shown in the fourth row of FIG. 2A. The deposited glucose acts as a barrier for the incoming dye flow and restricts its penetration towards the end of the evaporation pad. Hence, 4M (or less) glucose hydrogel appears to not be a suitable candidate for long term sampling with microneedles.

A control test was also conducted with just glycerogel in the osmotic patch, but without the microneedle patch. The glycerogel facilitates greater dye collection than 8M glycerin and 12M glycerin hydrogels, but lesser than with the presence of microneedles on both the pad and paper channel as shown in FIG. 2A and in the bar charts of FIGS. 3B and 3C. This additional dye quantity mainly comes from just the microneedle patch, which it samples after coming in contact with the model skin. The glycerogel withdraws dye from the microneedle patch as well as from the skin. Thus, presence of microneedle in between glycerogel and skin, aids with increased dye sampling on the paper channel. The operation of the whole device depends on the high osmotic pressure provided by the glycerogel.

The platform can be used to track the glucose metabolism in the body. The glucose levels in ISF correlate well with blood glucose. Maintaining a long-term record of ISF glucose can be useful, especially for diabetic patients. The platform can also be used to detect lactate levels in ISF. Lactate is an oxidative stress biomarker which depends on the intensity of exertion that the body goes through. A similar analysis can also be performed for monitoring drug levels in the body. The platform can also be applied to study plant health. The combination of glycerogel and microneedles can be used to withdraw pathogen DNA from leaves and stems. The monitoring platform can also be configured for continuous sensing by integrating enzymatic electrochemical sensors. Such a platform can deliver real-time and continuous monitoring of different ISF biomarkers, as will be discussed.

Wireless Electrochemical Sensing System

Wearable and wireless monitoring of biomarkers in ISF can provide a deeper understanding of a subject's metabolic stressors, cardiovascular health, and physiological response to, e.g., exercise. Here, a continuous monitoring platform combining a hydrogel for osmotic-microneedle extraction, with a paper microfluidic channel for facilitating ISF transport and management, a screen-printed electrochemical sensor, and a custom-built wireless wearable potentiostat system. Such a system can also be used for ISF extraction and monitoring of biomarkers. Osmosis enables zero-electrical power ISF extraction at rest, while continuous evaporation at the end of a paper channel allows collection of analytes. The positioning of the sensors provides near-instantaneous sensing at low fluid volume, and the custom designed potentiostat supports continuous monitoring with ultra-low power consumption. Overall, this wearable system can provide comprehensive and long-term continuous analysis of ISF biomarker trends in the human body during rest and exercising conditions.

One example of ISF sensing can be for tissue and systemic lactate is often a byproduct of anaerobic glycolysis, commonly generated as an outcome of oxidative stress experienced by the body due to high levels of physical exertion. It is an important biomarker for assessing functional health of muscles and physiological response to exercise. Lactate is present in ISF. Lactate is also present in the blood where its concentration ranges from 0.5 to 2 mM for healthy subjects at rest. Greater amounts of lactate begins to appear in the blood and ISF only when the body experiences high intensity exertion (>120 W) and crosses the lactate threshold (LT) workout intensity. For this reason, the blood lactate concentration can peak up to about 11 mM upon surpassing LT.

Most state-of-the-art wearable sensors determine the lactate content using either colorimetry or electrochemistry. The biorecognition elements in these sensors typically use either lactate oxidase (LOₓ) or lactate dehydrogenase (LDH) enzymes. For wearable colorimetric platforms, the biofluid is allowed to flow through serpentine microfluidic channels with pre-immobilized lactate specific enzymatic assays. The incoming biofluid interacts with the assay to form a colored compound whose intensity directly correlates with the amount of lactate. Wearable electrochemical lactate sensors are typically operated via amperometry where the enzyme is immobilized on the working electrode for biorecognition. The lactate undergoes an enzymatic redox reaction to generate a mediated change in current, whose magnitude directly correlates with the amount of lactate. Other techniques that could quantify lactate concentrations include chemiluminescence, electrochemiluminescence, and spectroscopy.

Electrochemical lactate sensing platforms based on various biological fluids offer higher sensitivity and linear dynamic range (LDR), and a lower limit of detection (LOD, ˜nM range), as compared to their colorimetric counterparts. Moreover, electrical signals generated from electrochemical sensors can directly interface traditional analog electronics. The skin-interfacing electrodes of these electrochemical lactate sensors can be fabricated via photolithography, screen printing, sputter coating, inkjet printing, wax printing or direct stamping techniques. These electrodes could be printed on sheets of either polyimide (PI), polyethylene terephthalate (PET), polyethylene naphthalate (PEN), plastic, polycarbonate (PC), polyester (PE) or tattoo paper. Thus, the existing devices are thin, flexible, and could easily adhere onto the skin surface.

The ink material of the working and counter electrodes can be either carbon, platinum, silver or silk, while the reference electrode is usually Ag/AgCl paste. The biorecognition element, which could be LOₓ or LDH enzyme in bovine serum albumin (BSA) or phosphate buffer saline (PBS) is immobilized on the working electrode. The enzyme is crosslinked with glutaraldehyde, and sometimes mixed with carbon nanotubes, graphene, platinum, or silver nanoparticles to enhance the sensitivity towards lactate detection by increasing the electrochemically active area. Moreover, either chitosan or Nafion solutions can also be added on the electrode to remove the interference from other analytes, and control the oxygen and analyte diffusion towards the electrode surface. The LOD and LDR of these sensors can range between 0.2-100 nA·mm⁻²·mM⁻¹ and 0-30 mM, respectively.

It is based on a zero-power, ISF extraction technique, which deploys osmosis, microfluidics, and microneedles simultaneously. The platform comprises four components: (1) a hydrogel having a higher osmotic strength than the hydrated microneedles, (2) an electrochemical sensor, (3) a paper microfluidic channel, and (4) a custom, low-power, flexible wireless potentiostat system.

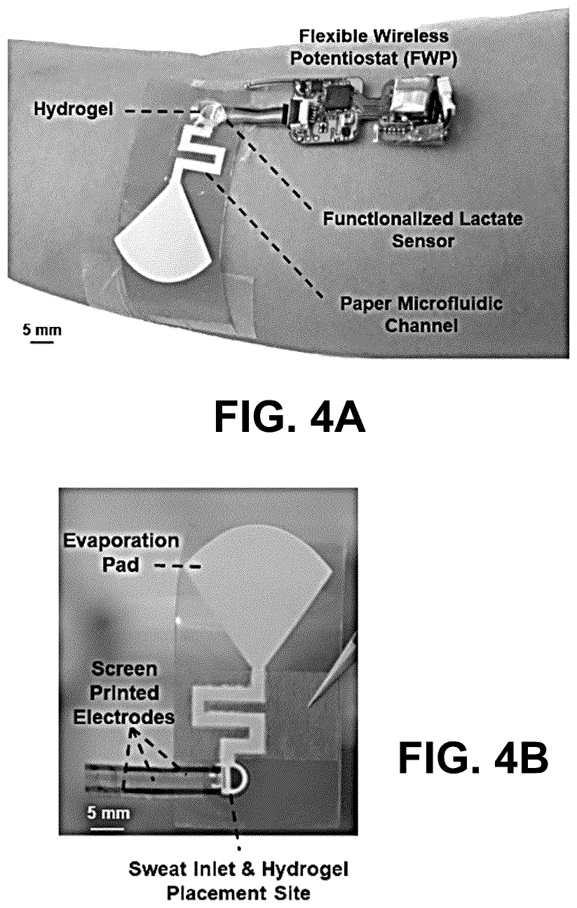

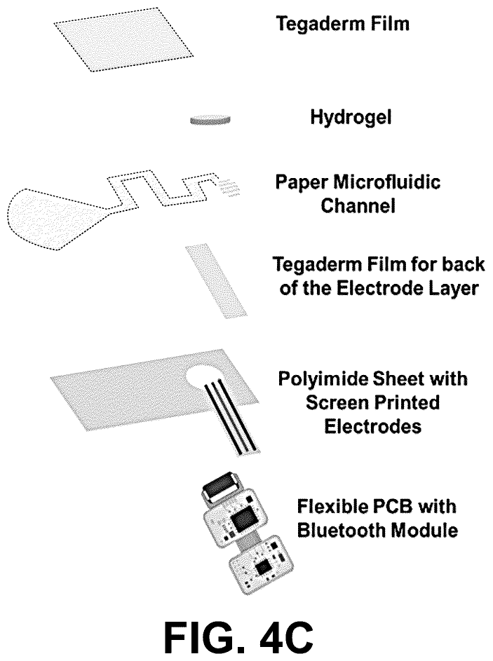

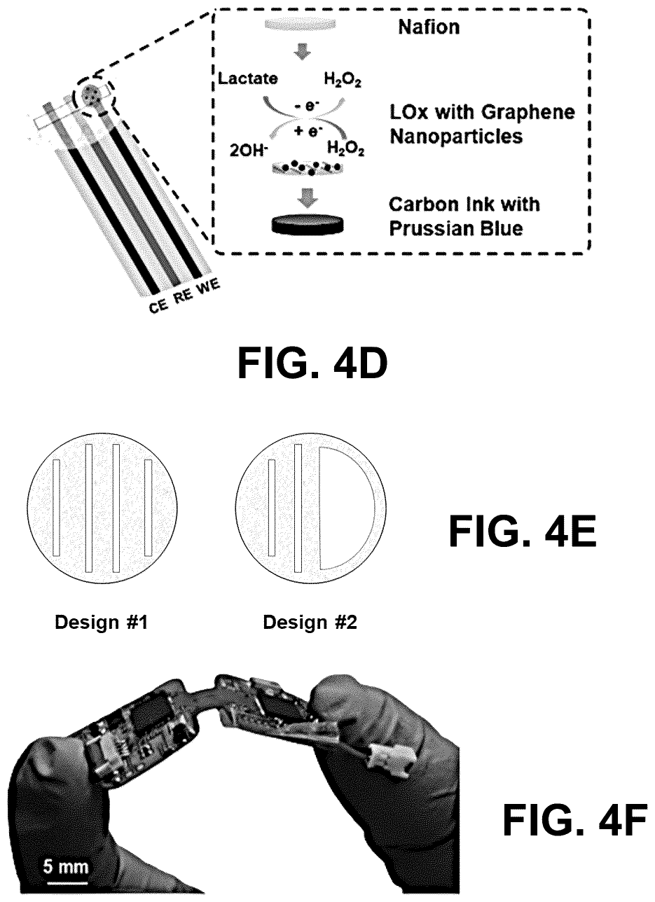

Design Aspects and Working Principle. The platform integrates enzymatic electrochemical sensors and the FWP to the wearable osmotic microneedle extraction technique. FIGS. 4A-4F illustrate an example wearable for continuous biochemical monitoring. FIGS. 4A and 4B show images of the system attached to the forearm, and on the bench (without hydrogel), respectively. The image of FIG. 4A shows the integrated system comprising an osmotic hydrogel, a paper microfluidic channel, a functionalized sensor (e.g., functionalized lactate sensor), and flexible wireless potentiostat (FWP) mounted on the forearm using an adhesive patch. FIG. 4B is a detailed view of the arrangement of the functionalized sensor with the paper microfluidic channel. The sensing electrodes (e.g., lactate sensing electrodes or other functionalized sensing electrodes) can be placed in between the hydrogel and the circular section of the paper channel. Overall, the platform is thin, flexible, and can be easily mounted on the skin using either a Velcro strap or a skin-compatible adhesive as shown in FIG. 4A. The functionalized lactate sensor with integrated hydrogel and paper channel weighs about 165 mg and has dimensions of 70×30×0.02 mm.

FIG. 4C is an exploded view showing a schematic of the individual platform components. The PI sheet is initially cut out to create a circular space for placing the hydrogel osmotic patch into and to interface the electrodes with the inlet (circular section) of the paper channel. The working electrode can contain, e.g., Prussian blue (PB, electroactive species) and can be functionalized with a solution of LOₓ enzyme, graphene nanoparticles, and Nafion. FIG. 4D is a schematic diagram highlighting the functional components of the working electrode and the reaction mechanism of lactate detection. The circular end of the paper channel is then inserted into the circular space on the PI sheet, such that the exposed surface of the electrodes resides directly above the inlet of the paper channel.

The paper channel design used in this study is different in three aspects: (1) The circular inlet of the paper channel in this study has two slits and a semi-circular section cut out. FIG. 4E illustrates examples of circular sections of the paper channel. Such a design was chosen to ensure that a lower volume of ISF was sufficient to saturate the lesser surface area of paper and more quickly reach the electrodes. This arrangement has proven to be advantageous during the trials at rest where the extracted ISF volume is extremely low (about 1-2 μL). The paper area can be varied to achieve the desired operation as illustrated in FIG. 4E. (2) The shape of the rectangular section of the paper channel was modified from a straight channel to a tortuous path. This increased (by about 90%) the net surface area of the paper channel in between the circular end and evaporation pad

A layer of commercially available thermoplastic polyurethane soft film (e.g., Tegaderm®, 3M Company, Saint Paul, MN, USA) was applied on top of the tortuous rectangular section of the paper, and over the printed electrodes (except for the circular hole on the PI sheet). Covering the tortuous channel section prevents evaporation from the region between the hydrogel and evaporation pads. The electrodes are also covered with a Tegaderm® layer to ensure that they do not directly touch the skin surface once mounted on the forearm. Hence, lactate will be sensed only at the inlet of the paper channel. The hydrogel is then placed on the circular hole, such that the three electrodes lie in between the hydrogel and paper. A Tegaderm® layer is applied on top of the hydrogel to position it firmly over the electrodes. The whole system is then connected to the custom FWP with a spacer to ensure secure connection and is mounted on the forearm for testing.

The functionality of the system is based on the simultaneous action of three basic effects to deliver long-term ISF extraction, collection, and sensing: osmosis, capillary wicking, and evaporation. Osmosis is the main pumping mechanism for ISF extraction. A hydrogel infused with a highly concentrated glucose (or glycerin) solution (osmolyte) generates the osmotic driving force from the microneedles. This hydrogel is placed in the circular chamber of platform as shown in FIGS. 4A-4C. The hydrogel disk and the circular end of the paper conduit directly interface the microneedles. The hydrogel withdraws ISF sample due to its higher osmotic strength with respect to the fluid inside the microneedles.

The lactate in the withdrawn fluid sample can then be sensed on the circular section of the paper microfluidic channel, where it creates an electrochemical cell across the working, counter, and reference electrodes (ELS, FIG. 4D) on the PI sheet or it can be transported to another location by the paper microfluidic for analysis.

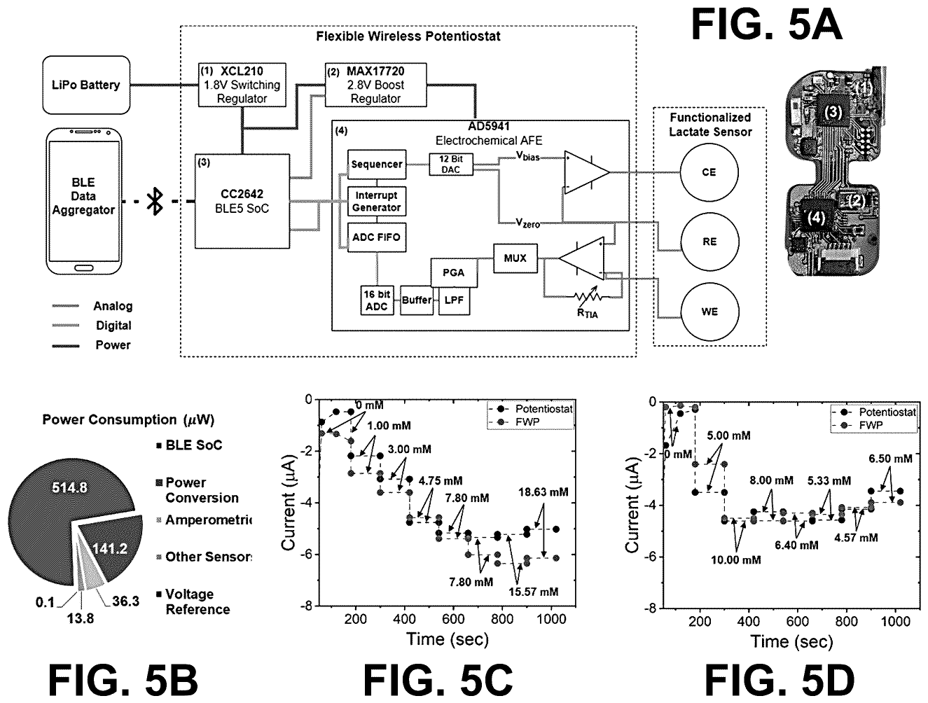

The signal transduction, measurement, and data transmission portion of the platform was designed as a wireless flexible wearable potentiostat (FWP) for increased wearability. FIG. 5A is a block diagram and corresponding labeled circuit board for the FWP, which was fabricated on a flexible circuit board, with dimensions of 45 mm×19 mm×2 mm and a weight of about 1.27 g without a battery. Amperometric lactate data were measured using an electrochemical analog front end (e.g., AFE, AD5941, Analog Devices, Cambridge, MA, USA), which controls the bias of the amperometric cell. The AFE contains internal memory which is used to aggregate the data. Once 10 sample readouts are aggregated, the amperometric data can be read by a wireless system on chip (e.g., SoC, CC2642, Texas Instruments, Dallas, TX, USA) and then wirelessly transmitted using the Bluetooth Low Energy (BLE) protocol to a nearby smart mobile device (e.g., Apple iPad) running a custom data aggregation app.

The application can also be used to change the bias of the amperometric cell, the gain of the transimpedance amplifier, and the sampling rate. This enables future use of the FWP with sensors functionalized for other target analytes. The gain of the system was set at 100 kΩ for a current resolution of 835 pA and a current range of ±9.0 μA. This value was chosen to balance sensitivity and dynamic range over the expected lactate concentrations. The limit of detection (3× the standard deviation of noise) of the amperometric front end was found to be 150 pA at 512 kΩ gain. The ELS interfaces to the electrochemical AFE on the FWP via a 5-pin FFC connector, such that the FWP can be reused, and the ELS can be replaced as needed.

As the system functions with zero-power osmotic-microneedle ISF extraction, the only electrical power consumed by the system is from the wearable electronics. Coupling osmotic-microneedle ISF extraction with a low-power electronic sensing system provides a significant decrease in power consumption, leading to a substantial increase in battery life, which is a key concern for wearable system development.

FIG. 5D illustrates an example of benchtop potentiostat vs. FWP output with the functionalized lactate sensor in dynamically increasing and decreasing lactate concentrations.

A wearable, continuous monitoring platform based on a zero-power sampling patch has been presented. The platform can be constructed on a thin polyimide sheet using a hydrogel or glycerogel containing high solute concentration acting as the pump for fluid (e.g., ISF) withdrawal via microneedles and osmosis. The collected sample can be processed and analyzed via a paper microfluidic channel with an extended site for evaporation at the end. The sensor can be constituted by, e.g., screen-printed electrodes with immobilized LOₓ enzyme for lactate sensing, which are located at the site of the fluid inlet. The platform can be connected to a wireless flexible circuit system for on-board electrochemical analysis and real-time remote monitoring of fluid lactate or other biomarkers. The osmotic patch (e.g., hydrogel or glycerogel), paper channel, and the electrodes can be arranged in a way as to allow rapid sensing of lactate from skin (or other ISF biomarkers), even with low fluid volumes. In-vitro testing of the lactate sensors showed a LOD, LDR, sensitivity, and stability of 350 nM, 15 mM, 90 nA·mM⁻¹·mm⁻², and 4 days, respectively.

### Experimental Information

Hydrogel Synthesis. The hydrogels for the osmotic interface were made using acrylamide monomer, N—N′ methylenebisacrylamide as crosslinker and 2-hydroxy-4′-(2-hydroxyethoxy)-2-methylpropiophenone as photo-initiator. The monomer solution contained 22% (w/w) acrylamide, 0.48% (w/w) crosslinker and 0.15% (w/w) photo-initiator. The solution was cured inside a circular Petri dish (47 mm diameter) under a 175 mW/cm² UV lamp (Sunray 400-SM) for three minutes. Disks of 6 mm diameter were punched out and stored in an aqueous solution of 4M glucose for 24 hours. The 4M glucose solution served as the osmolyte since it led to the highest accumulation of maximum model biomarker on paper in our previous in-vitro experiments. After 24 hours, the infused disks were transferred to a fresh solution in a vial and stored for further usage. A single disk was taken out, blotted with a paper napkin, and was fitted inside the circular hydrogel pad holder before usage (FIGS. 4B and 4C).

Fabrication Preparation of Lactate Sensors: The lactate sensor was fabricated on a 25 μm thick polyimide sheet. Initially, a circular section (exactly as the diameter of the hydrogel) with space for the three-electrodes was cut out from the sheet using a CO 2 laser cutter (Universal Laser System VLS 3.5). Working electrode (WE, carbon ink with Prussian blue mediator), reference electrode (RE, Ag/AgCl), and counter electrode (CE, carbon ink) were then screen-printed one by one on the sheet, with a 230-mesh size mask. The dimensions of all the electrodes were 1.87×2.0 mm². The electrodes were cured at 80° C. for 10 minutes after screen printing. The individual sensor strips were then cut out using a laser cutter. 15 mg/mL graphene stock solution was prepared in ethanol and sonicated in the ultrasonic bath for an hour. 5 μL of an aqueous suspension (4 μL of 50 mg/mL LOₓ in 1×PBS and 1 μL of 15 mg/mL graphene in 1× PBS) was drop-casted on the WE and dried at 4° C. for 1 hour. 1 μL of 0.5 wt % Nafion was then drop-casted on the WE and dried at 4° C. for 1 hour. This concluded the enzyme immobilization process and lactate sensor fabrication.

Preparation of Paper Microfluidic Channel: The paper channel design used in platform was slightly modified for the application. The circular interface (osmotic pump region between the skin and hydrogel) of the paper has two slits and a semi-circular section cut out as shown in FIG. 4E. Moreover, the evaporation pad area of the paper channel was increased, while the rectangular section was modified to have a tortuous path, in comparison to the linear design in FIG. 1. The paper channel was placed on the PI sheet with electrode heads, making sure that all three electrodes lay above the slits on the circular end of the paper channel, upon interfacing. The rectangular section of the paper channel was covered with a Tegaderm® layer (1.9×2.9 cm²) to conceal it from evaporation. A Tegaderm® layer (1.5×0.6 cm²) was attached over the electrodes to prevent them from touching the skin during human trials. A piece of Kapton tape (0.6×0.6 cm²) was attached on the lower backside of the electrodes so that the sensors have a rigid connection with the flexible board. Finally, the hydrogel was placed on the circular end of the sheet. Another Tegaderm® layer (from the side of the circular hole in the prototype to the middle to the tortuous rectangular section) can be attached to seal the hydrogel and fix its position over the sensors and paper. This concluded the fabrication process. The prototype was always stored at 4° C. before any test. The lactate sensor and paper microfluidic channel portion of the platform weighs about 165 mg (before hydrogel addition) and has dimensions of 70 mm×30 mm×0.02 mm.

Development of FWP Electronics. The custom FWP system can be controlled by a Bluetooth Low Energy (BLE) SoC (CC2642, Texas Instruments), which interfaces with an electrochemical analog front end (AFE, AD5941, Analog Devices) containing the bias and transimpedance amplifier circuitry required for amperometric sensing. The electrochemical AFE can be programmed to measure lactate concentration from the 3-electrode electrochemical cell on the platform, which was connected via a 5-pin ribbon connector (Hirose). The sampling rate, bias voltage, and gain of the electrochemical cell can all be controlled by the SoC. After initialization, the AFE reads from the electrochemical cell, and stores amperometric current data into a buffer which can then be read by the SoC and transmitted over BLE.

The wearable electronic system was designed to run from a 3.7V lithium polymer battery. The battery voltage is regulated to 1.8V to power the BLE SoC and then boosted to 2.8V to power the electrochemical analog front end. A general diagram of the signal conditioning, wireless transmission, and power regulation circuitry can be found in FIG. 5A. The printed circuit board was fabricated on a 0.23 mm flexible polyimide substrate. For use during the exercise conditions, the system was coated with silicone and integrated with an adhesive patch (Upright).

The aggregated data can be transmitted over Bluetooth communication link to either a computing device (e.g., smartphone, tablet, laptop, etc.) running a custom Python script or a custom iOS app, in which the user can quickly set the gain and start the system for lactate measurement or input a custom Bluetooth command for control over more of the attributes of the amperometric sensing configuration. Both allow the user to control the bias, sampling rate, and gain of the system, allowing for use at a variety of operating points. The custom wearable electronic system can also be capable of measuring in a duty cycle mode, where the system can periodically halt measurement and remove the bias voltage from the electrochemical cell to prolong enzyme lifetime of functionalized electrodes and reduce power consumption. However, as the duty cycle test trends were similar to the regular chronoamperometry trends with the potentiostat during in-vivo testing at rest for 2 hours, it was decided to use chronoamperometry for all of the in-vitro and in-vivo tests for the benefits of higher temporal resolution.

FIG. 4E schematically illustrates two designs of the circular section of the paper channel. Design 2 has a smaller surface area than design 1. Hence, the fluid would travel relatively quicker in design 2 before entering the rectangular section of the paper channel. This also means more inflow of lactate over time in design 2. In-vivo chronoamperometric studies at rest with the benchtop potentiostat validated the same outcome (more negative current) for design 2.

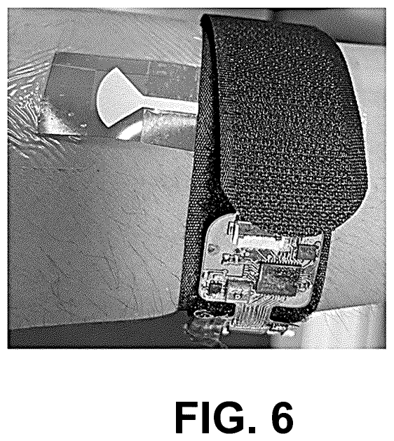

FIG. 6 is an image of a wearable electrochemical sensing system including the patch with a paper microfluidic channel with a smaller evaporation pad.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A microfluidic monitoring platform, comprising:
a microneedle patch comprising a plurality of microneedles on a first side;
an osmotic patch disposed on a second side of the microneedle patch opposite the plurality of microneedles, the osmotic patch comprising glycerogel, hydrogel, or solid electrolyte, equilibrated with an osmolyte; and
a microfluidic or fluid transport film or material channel disposed between the osmotic patch and the microneedle patch, the microfluidic or fluid transport film or material channel configured to extract and transport fluid from the microneedle patch and osmotic patch complex.

2. The microfluidic monitoring platform of claim 1, wherein the osmolyte is glycerin or glucose.

3. The microfluidic monitoring platform of claim 1, wherein the osmolyte of the glycerogel comprises pure glycerin.

4. The microfluidic monitoring platform of claim 1, wherein the fluid is interstitial fluid (ISF) or extracellular fluid (ECF).

5. The microfluidic monitoring platform of claim 1, wherein the microfluidic or fluid transport film or material channel comprises an extraction portion, a transport portion, and an evaporation portion.

6. The microfluidic monitoring platform of claim 5, wherein the extraction portion comprises a circular paper pad.

7. The microfluidic monitoring platform of claim 6, wherein the circular paper pad comprises a pattern of openings.

8. The microfluidic monitoring platform of claim 5, wherein the transport portion comprises a rectangular section extending between the extraction portion and the evaporation portion.

9. The microfluidic monitoring platform of claim 8, wherein the rectangular section comprises a linear path or a tortuous path.

10. The microfluidic monitoring platform of claim 8, wherein the evaporation portion comprises an evaporation pad at an end of the rectangular section.

11. A wearable electrochemical sensing system, comprising:
a microfluidic monitoring platform comprising:
an osmotic patch comprising glycerogel or hydrogel;
a microfluidic or fluid transport film or material channel configured to extract fluid through the microneedle patch via capillary wicking; and
at least one sensor disposed between the osmotic patch and the microfluidic or fluid transport film or material channel; and
processing circuitry coupled to the at least one sensor, the processing circuitry configured to monitor presence of a chemical or biomarker in the fluid based upon signals obtained from the sensor.

12. The wearable electrochemical sensing system of claim 11, wherein the at least one sensor comprises functionalized sensing electrodes.

13. The wearable electrochemical sensing system of claim 10, wherein the fluid is interstitial fluid (ISF) or extracellular fluid (ECF).

14. The wearable electrochemical sensing system of claim 10, wherein the hydrogel is equilibrated with glycerin or glucose.

15. The wearable electrochemical sensing system of claim 10, wherein the processing circuitry comprises potentiostat circuitry.

16. The wearable electrochemical sensing system of claim 10, wherein the processing circuitry comprises a communications interface configured for wirelessly communicate sensor information to a user device.

17. The wearable electrochemical sensing system of claim 10, wherein the microfluidic or fluid transport film or material channel comprises an extraction portion, a transport portion, and an evaporation portion.

18. The wearable electrochemical sensing system of claim 17, wherein the extraction portion comprises a circular paper pad including a pattern of openings.

19. The wearable electrochemical sensing system of claim 18, wherein electrodes of the at least one sensor are located between the openings of the circular paper pad.

20. The wearable electrochemical sensing system of claim 18, wherein the transport portion comprises a rectangular section having a tortuous path.
